(19) United States
(12) Patent Application Publication
(10) Pub. No.: US 2019/0228179 A1
(43) Pub. Date: Jul. 25, 2019

(54) CONTEXT-BASED ACCESS TO HEALTH INFORMATION

(71) Applicant: International Business Machines Corporation, Armonk, NY (US)

(72) Inventors: Sarbajit K. Rakshit, Kolkata (IN); Martin G. Keen, Cary, NC (US); James E. Bostick, Cedar Park, TX (US); John M. Ganci, JR., Cary, NC (US)

(21) Appl. No.: 15/878,673

(22) Filed: Jan. 24, 2018

(51) Int. Cl. G06F 21/62; G16H 10/60; G16H 40/67

(52) U.S. Cl. CPC G06F 21/6245; G16H 40/67; G16H 10/60

(57) ABSTRACT

A computer system processes requests for health information. A request for health information of an entity is received from a requester, wherein a set of access restrictions is associated with the health information. A context of the request is determined based on one or more factors, and the request is processed to retrieve health information appropriate for the requester based on the associated set of access restrictions and the determined context of the request. Embodiments of the present invention further include a method and computer program product for processing requests for health information in substantially the same manner described above.

### BACKGROUND

Present invention embodiments relate generally to the field of health information, and more specifically, to accessing health information based on contextual factors.

In the field of health information, wearable devices such as smart watches and fitness trackers have a variety of health-monitoring components by which personal health information may be collected. For example, a wearable device can track a user's number of steps taken, heart rate, sleep history, and the like. While there are many reasons to share health information, privacy concerns may restrict the sharing of this information.

### SUMMARY

According to one embodiment of the present invention, a computer system processes requests for health information. A request for health information of an entity is received from a requester, wherein a set of access restrictions is associated with the health information. A context of the request is determined based on one or more factors, and the request is processed to retrieve health information appropriate for the requester based on the associated set of access restrictions and the determined context of the request. Embodiments of the present invention further include a method and computer program product for processing requests for health information in substantially the same manner described above.

### BRIEF DESCRIPTION OF THE DRAWINGS

Generally, like reference numerals in the various figures are utilized to designate like components.

FIG. 1 is a block diagram depicting a computing environment for health information sharing, in accordance with an embodiment of the present invention;

FIG. 2 is a flow chart depicting operations for context-based health information sharing, in accordance with an embodiment of the present invention;

FIG. 3 is a block diagram depicting an example implementation of restricted sharing of health information, in accordance with an embodiment of the present invention;

FIGS. 4A and 4B are block diagrams depicting examples of context-based health information sharing, in accordance with an embodiment of the present invention; and

FIG. 5 is a block diagram depicting a computing device, in accordance with an embodiment of the present invention.

### DETAILED DESCRIPTION

Present invention embodiments relate generally to the field of health information, and more specifically, to sharing health information based on contextual factors. Using wearable devices, individuals may monitor and collect various metrics regarding their health. In some instances, individuals may wish to share their health information with others; for example, participants in a group exercise session may desire to share with the other participants their heart rate during the session. However, wearable devices may also collect information that a user would desire to keep private from others. Aspects of embodiments of the present invention intelligently process requests for health information on the basis of the context of the request while also restricting some information.

It should be noted that references throughout this specification to features, advantages, or similar language herein do not imply that all of the features and advantages that may be realized with the embodiments disclosed herein should be, or are in, any single embodiment of the invention. Rather, language referring to the features and advantages is understood to mean that a specific feature, advantage, or characteristic described in connection with an embodiment is included in at least one embodiment of the present invention. Thus, discussion of the features, advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention may be practiced without one or more of the specific features or advantages of a particular embodiment. In other instances, additional features and advantages may be recognized in certain embodiments that may not be present in all embodiments of the invention.

These features and advantages will become more fully apparent from the following drawings, description and appended claims, or may be learned by the practice of the invention as set forth hereinafter.

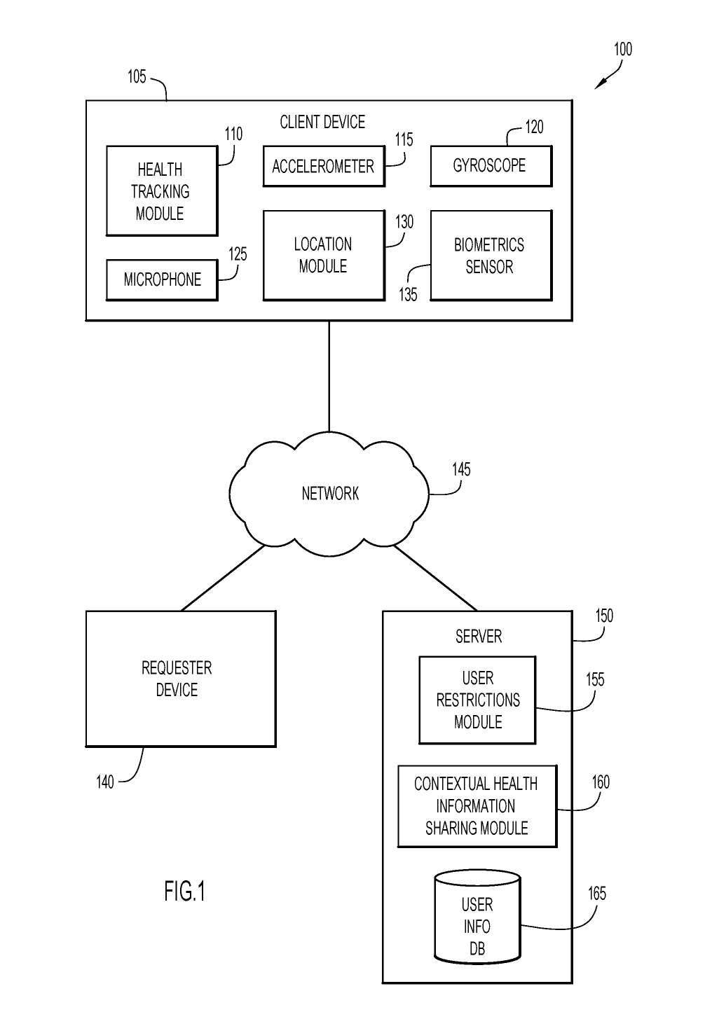

Present invention embodiments will now be described in detail with reference to the Figures. FIG. 1 is a block diagram depicting a computing environment 100 for health information sharing, in accordance with an embodiment of the present invention. As depicted, computing environment 100 includes client device 105, requester device 140, network 145, and server 150. Client device 105 includes health tracking module 110, accelerometer 115, gyroscope 120, microphone 125, location module 130, and biometrics sensor 135. Server 150 includes user restriction module 155, contextual health data sharing module 160, and user information database 165. It is to be understood that the functional division among components of computing environment 100 have been chosen for purposes of explaining embodiments of the present invention and is not to be construed as a limiting example.

Client device 105 may include any device capable of monitoring and sharing health information regarding a user. Client device 105 may be a wearable device, such as an activity tracker, smart watch, fitness band, pedometer, etc. Depending on the type of wearable device, client device 105 may be worn around a person's wrist, arm, ankle, torso, neck, worn in a pocket, or otherwise kept on their person. Client device 105 may track a user's health via health tracking module 110, which analyzes input from one or more sensors to produce quantitative and/or qualitative health information.

Client device 105 may be associated with one or more sensors for obtaining health data from a user, such as accelerometer 115, gyroscope 120, microphone 125, location module 130, and biometrics sensor 135. Accelerometer 115 may include any sensor that measures acceleration in one or more dimensions. Gyroscope 120 may include any sensor that measures orientation and angular velocity. By analyzing data gathered from accelerometer 115 and gyroscope 120, health tracking module 110 may calculate motion information about a user, such as how many steps the user has taken, whether the user is walking or running, and number of calories burned.

Microphone 125 may include any transducer that converts sound into an electrical signal. Microphone 125 may assist health tracking module 110 in determining the emotional state of a user by measuring the user's speech.

Location module 130 may include any device capable of determining the location of client device 105. Location may include one or more of latitude, longitude, and elevation. In one embodiment, location module 130 uses a global positioning system in order to determine location. In another embodiment, location module 130 uses triangulation to determine location. Health tracking module 110 may determine information about a user's motion by tracking location of client device 105 over time.

In general, biometrics sensor 135 may include any sensor capable of collecting and measuring biosignals. Biosignals are any electrical or non-electrical signals in living organisms that can be measured and monitored. Biometrics sensor 135 may include one or more of a heart rate monitor, blood pressure monitor, temperature monitor, pulse oximeter, and skin conductance sensor. Thus, biometrics sensor 135 may measure a user's heart rate, blood pressure (systolic and diastolic), temperature, oxygen saturation, and electrodermal activity.

Health tracking module 110 may analyze data collected by the various sensors associated with client device 105 to determine health information about the user. For example, a user's pulse may be determined from the heart rate monitor, and a user's blood pressure may be determined from the blood pressure monitor. As there is a correlation between skin temperature and stress, a user's stress level may be determined from the temperature monitor. Health tracking module 110 may track a user's sleep history using a combination of accelerometer 115, gyroscope 120 and the heart rate monitor of biometrics sensor 135.

Furthermore, the emotional state of a user may be determined by analyzing biometrics such as heart rate variability (from the heart rate monitor), movement patterns (from accelerometer 115 and gyroscope 120), and frequency of speech (from microphone 125). Health tracking module 110 may also use data from microphone 125 to analyze speech content, pitch, and loudness of speech in order to evaluate a user's emotional state. For example, speech that is higher in pitch or louder than a threshold (such as a user's typical baseline range) may indicate that the user is upset or angry. A lower heart rate, infrequency of motion, or a quieter speech volume may indicate that a user is relaxed.

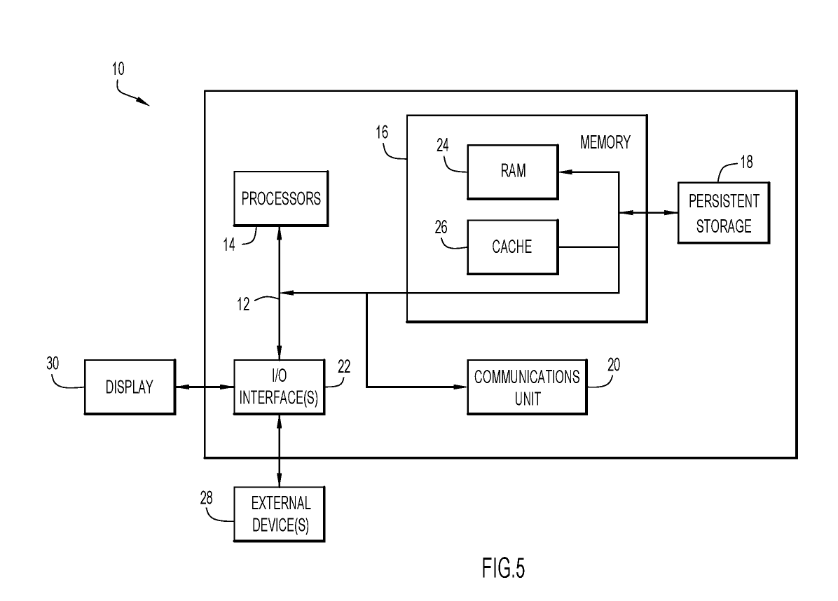

Requester device 140 may include any device by which an individual may request health information from client device 105 or server 150. In various embodiments of the present invention, requester device 140 maybe a laptop computer, a tablet computer, a netbook computer, a personal computer (PC), a desktop computer, a personal digital assistant (PDA), a smart phone, a thin client, or any programmable electronic device capable of executing computer readable program instructions. Requester device 140 may include internal and external hardware components, as depicted and described in further detail with respect to FIG. 5. Requester device 140 may include some or all of the capabilities of client device 105, such as being able to determine the location of a requester device 140, heart rate of a user of requester device 140, etc.

Network 145 can be, for example, a local area network (LAN), a wide area network (WAN) such as the Internet, or a combination of the two, and include wired, wireless, or fiber optic connections. In general, network 145 can be any combination of connections and protocols that will support communications between client device 105, requester device 140, and server 150 in accordance with an embodiment of the present invention.

Server 150 may include any device capable of intermediating context-based sharing of health information between client device 105 and requester device 140. In general, server 150 may receive a request from requester device 140 and, based on user restrictions and the context of the request, determine which relevant health information to retrieve from client device 105. In one embodiment, some or all of the tracking functions of health tracking module 110 are performed by server 150, which receives input from the various information-collecting modules of client 105 over network 145.

User restrictions module 155 may determine user restrictions on sharing health information, such as time restrictions, location restrictions, and requester restrictions. Requests for health information may be filtered first through user restrictions module before being processed by contextual health information sharing module 160. User restrictions module 155 may store and retrieve restriction information from user information database 165.

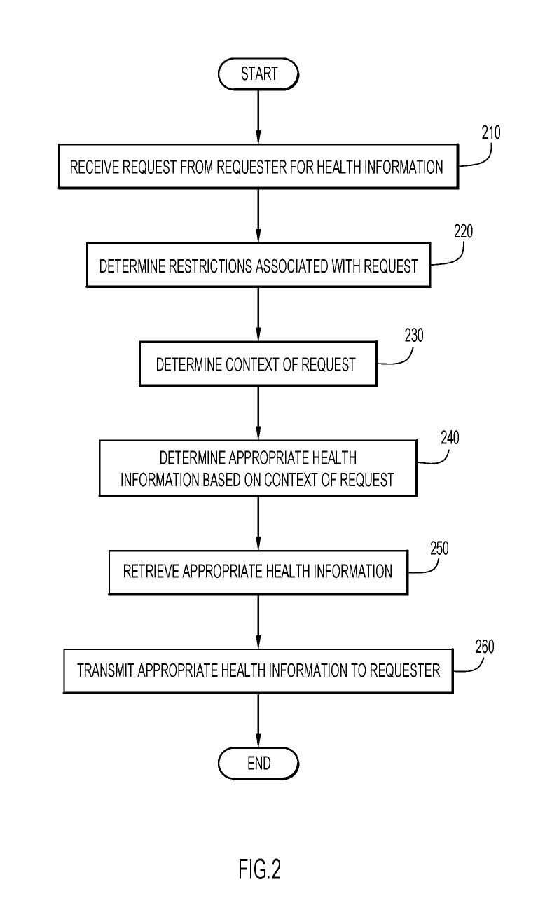

Contextual health information sharing module 160 may determine which health information collected from client device 105 is most appropriate or relevant to include in a response to a request for health information from requester device 140. Context factors may include the nature of the relationship of the requester and the user (e.g., friend, neighbor, employer, co-worker, cousin, spouse, etc.), the current time, the current activity of the user and/or requester, and the current location of the user and/or requester. These context factors will be described below in further detail with reference to FIG. 2.

User information database 165 may store information regarding users, such as a user's health information, relationships to other users, restrictions on other users, date and time restrictions, and the like. User information database 165 may include any non-volatile storage media known in the art. For example, user information database 165 can be implemented with a tape library, optical library, one or more independent hard disk drives, or multiple hard disk drives in a redundant array of independent disks (RAID). Similarly, data on the database may conform to any suitable storage architecture known in the art, such as a file, a relational database, an object-oriented database, and/or one or more tables.

FIG. 2 is a flow chart 200 depicting operations for context-based health information sharing, in accordance with an embodiment of the present invention.

A request for information is received at operation 210. This may include server 150 receiving a request from requester device 140 for health information from client device 105. The requesting individual may pose a question by selecting one or more pre-determined requests for health information. For example, a user of requester device 140 may select an option “how is user A doing?” or “how many steps has user A taken today?” The requester may select a user on a mobile app, select the user on a health web site, or make a selection using their own wearable device. In one embodiment, a user of requester device 140 inputs a request to requester device verbally, or inputs a text-based request in ordinary language; techniques such as natural language processing are then applied to determine the health information that the requester is requesting. Requester device 140 may be scheduled to place recurring requests according to conditions such as time, location of user, user activity, etc.

Restrictions associated with the request are determined at operation 220. This may include user restrictions module 155 processing the request against a set of access restrictions. These access restrictions may be a set of rules that define which health information is shareable. Each access restriction may either be provided by a user or may be predetermined. The restrictions may be requester-specific; for example, a user may prevent a particular requester from ever obtaining some or all of their health information. A user may also create restrictions based on which health information they would like to restrict globally, such as restricting the sharing of their emotional state information. In some embodiments, the access restrictions include restriction on when health information can be shareable, such as during a particular time frame or at a specific location. Furthermore, access restrictions may include any combination of requester-based restrictions, time-and-place-based restrictions, and health information-based restrictions.

In one example, a set of restrictions may restrict a user's health information such that only their heart rate is shareable, and even then, only shareable to their workout partner while they are both at the gym on Tuesdays and Thursdays. In such case, a request made by the workout partner for health information other than heart rate while away from the gym on a Wednesday may be restricted (since it violates every specified restriction in this particular example). Access restrictions may also whitelist or blacklist requesters; for example, a user may choose to make all of the health information shareable with their spouse, guardian, emergency contact, or physician, or may choose to block somebody entirely. Some restrictions may be pre-determined based on how the user defines their relationship with the requester; for example, if the user selects “wife” then there may be fewer access restrictions than if the user selects “friend” or “co-worker.” Pre-determined access restrictions may also enable devices to comply with health regulations.

If a request is restricted entirely, server 150 may respond to requester device 140 with a predetermined response such as “access denied” or “health information unavailable,” etc. Alternatively, if a request for health information is restricted, but there is some other health information not restricted to requester device 140, then user restrictions module 155 and contextual health information sharing module 160 may treat the request as a request for whichever health information is shareable. For example, if a requester makes a request for a user's blood pressure, and the user has restricted the sharing of their blood pressure but not their heart rate, then user restrictions module 155 and contextual health information sharing module 160 may treat the request as a request for the user's heart rate.

The context of the request is determined at operation 230. This may include contextual health information sharing module 160 determining the context of the request based on one or more factors and subject to the user's access restrictions. Factors may include the requester's relationship to the user, the current time, the current activity in which user is engaged, the current location of the user and/or requester, or any combination thereof. In some embodiments, the context of the request is determined before restrictions are determined, whereas in other embodiments, restrictions are determined first.

The appropriate health information is determined at operation 240. This may include selecting which health information is most appropriate to share based on the context of the request and subject to the restrictions. For an example that illustrates how relationship statuses are relevant to context, a user's emotional state may be appropriate to share with the user's spouse, but not with their employer. Thus, even if an employer is not restricted from receiving a user's emotional state information, contextual health information sharing module 160 may determine that the user's stress level is a better response than the user's emotional state when an employer makes a request about the user. Similarly, if a user's exercise partner requests to see how the user is doing, the relevant context would be exercise, so the appropriate health information may include the user's heart rate or number of calories burned.

If a request for health information is made early in the morning, the context may be related to the user's sleep, so a response might include health information such as the amount of sleep as user has had or whether they have woken up yet. Requests made later in the day or evening may, for example, include a summary of the user's activity for the day rather than simply the user's current information.

The user's activity status may also be important in determining which health information is appropriate to include in a response. For example, if a user is participating in a sporting event or athletic competition, then heart rate and blood pressure may be deemed more appropriate than emotional state. A user's activity status may be determined based on information from client device 105 such as detection of jogging-like motion. For example, if accelerometer 115 and gyroscope 120 detect repetitive back-and-forward motion of the wrist, but the location of client device 105 is not changing substantially, then the user may be using a treadmill or stationary bicycle. In one embodiment, a user's activity status is inferred using location module 130; for instance, if a user is at a location that is known to have a gym, it may be assumed that the user is exercising. Client device 105 may integrate with a user's electronic calendar or scheduler so that activity status can be determined or even predicted based on the user's scheduled activities.

Location of the user and/or requester may be another factor that is relevant to the context of the request. Similarly to activity status, if a user's client device 105 reports a location near a gym, the context of the request may be exercise. If location module 130 indicates that the user is moving through a body of water, then the user may be swimming or rowing. Location of the requester may also be relevant; for example, if the user and the requester are in close proximity to each other, information such as mood or stress level may be more appropriate to share than if the individuals were not in each other's company.

The health information is retrieved at operation 250. This may include server 150 fetching the health information that contextual health information sharing module 160 determined to be appropriate. The health information may be retrieved from client device 105 or from user information database 165.

The health information is transmitted to the requester at operation 260. This may include server 150 replying to requester device 140 over network 145 with the appropriate health information. Server 150 may thus act as an intermediary between client device 105 and requester device 140, which may not communicate directly with each other.

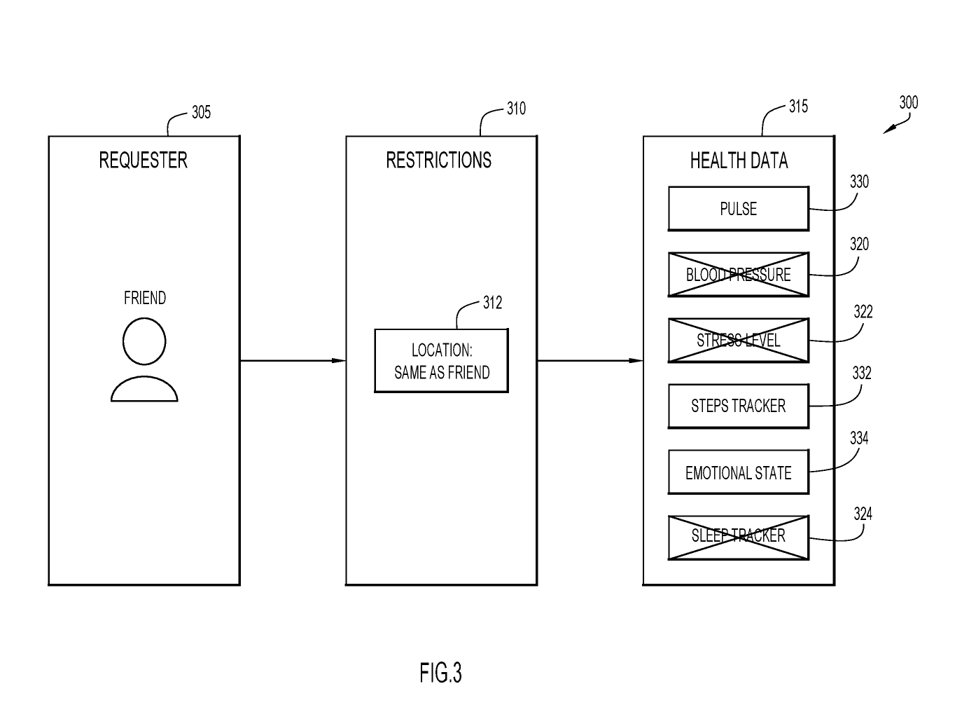

FIG. 3 is a block diagram 300 depicting an example implementation of restricted sharing of health information, in accordance with embodiments of the present invention. As depicted, FIG. 3 illustrates a requester 305 who is subject to several restrictions 310 with respect to accessing health data 315. In some embodiments, the nature of the relationship between a user and a requester, as well as any restrictions on the requester or health information, are identified when the user adds the requester as a contact for health information sharing. In the depicted example, the user's relationship to the requester is that they are friends. The friend who is requesting is restricted by a restriction 312 to only receiving health information when sharing a location as the user, and the requester is restricted from accessing blood pressure 320, stress level 322, or sleep tracker information 324 of health data 315. Contextual health information sharing module 160 may then choose among pulse 330, steps tracker 332, and emotional state 334 of health data 315 to select appropriate health information to include in a response.

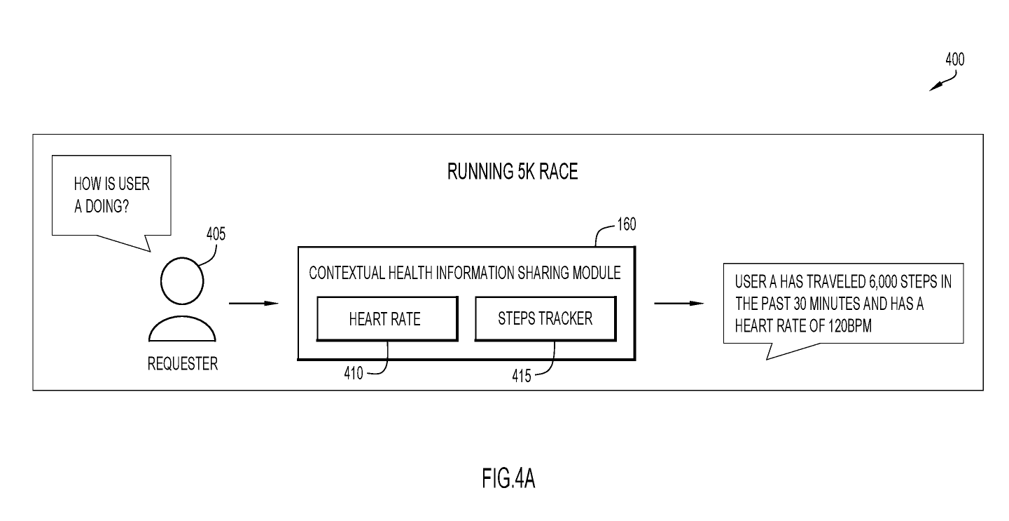

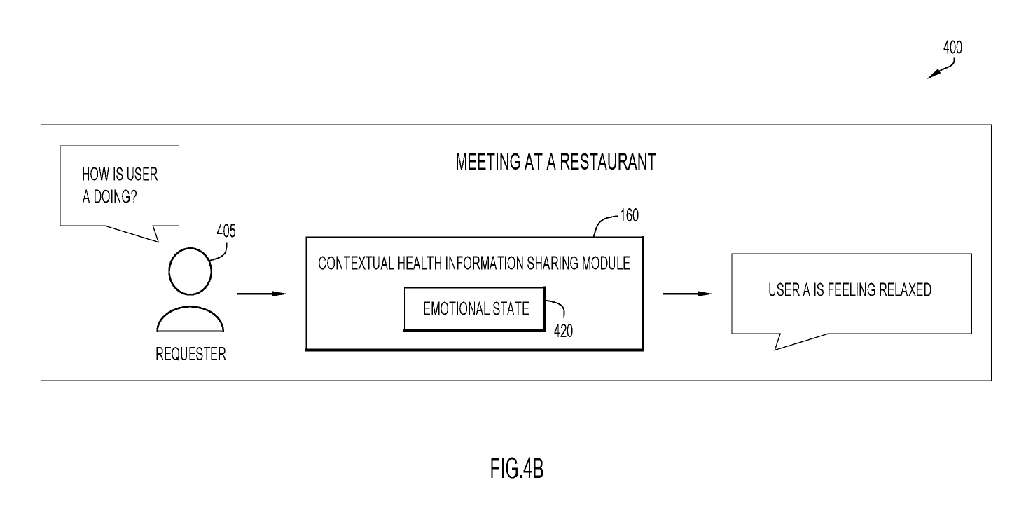

FIGS. 4A and 4B are block diagrams 400 depicting examples of context-based health information sharing, in accordance with embodiments of the present invention. In these examples, restrictions have already been determined, and contextual health information sharing module 160 may select which health information is most appropriate to share. FIG. 4A illustrates a requester 405 asking “how is user A doing” while user A is running in a race. In this context, contextual health information sharing module 160 may deem that heart rate 410 and steps 415 taken are appropriate to share with the requester. In contrast, FIG. 4B depicts the same requester 405 asking the same question in a different context. Here, the user is meeting the requester at a restaurant, so contextual health information sharing module 160 may select “emotional state” 420 as the appropriate health information to share.

FIG. 5 is a block diagram depicting components of a computer 10 suitable for executing the methods disclosed herein. Computer 10 may enable client device 105, requester device 140, and server 150 to provide context-based health information sharing in accordance with embodiments of the present invention. It should be appreciated that FIG. 5 provides only an illustration of one embodiment and does not imply any limitations with regard to the environments in which different embodiments may be implemented. Many modifications to the depicted environment may be made.

As depicted, the computer 10 includes communications fabric 12, which provides communications between computer processor(s) 14, memory 16, persistent storage 18, communications unit 20, and input/output (I/O) interface(s) 22. Communications fabric 12 can be implemented with any architecture designed for passing data and/or control information between processors (such as microprocessors, communications and network processors, etc.), system memory, peripheral devices, and any other hardware components within a system. For example, communications fabric 12 can be implemented with one or more buses.

Memory 16 and persistent storage 18 are computer readable storage media. In the depicted embodiment, memory 16 includes random access memory (RAM) 24 and cache memory 26. In general, memory 16 can include any suitable volatile or non-volatile computer readable storage media.

One or more programs may be stored in persistent storage 18 for execution by one or more of the respective computer processors 14 via one or more memories of memory 16. The persistent storage 18 may be a magnetic hard disk drive, a solid state hard drive, a semiconductor storage device, read-only memory (ROM), erasable programmable read-only memory (EPROM), flash memory, or any other computer readable storage media that is capable of storing program instructions or digital information.

The media used by persistent storage 18 may also be removable. For example, a removable hard drive may be used for persistent storage 18. Other examples include optical and magnetic disks, thumb drives, and smart cards that are inserted into a drive for transfer onto another computer readable storage medium that is also part of persistent storage 18.

Communications unit 20, in these examples, provides for communications with other data processing systems or devices. In these examples, communications unit 20 includes one or more network interface cards. Communications unit 20 may provide communications through the use of either or both physical and wireless communications links.

I/O interface(s) 22 allows for input and output of data with other devices that may be connected to computer 10. For example, I/O interface 22 may provide a connection to external devices 28 such as a keyboard, keypad, a touch screen, and/or some other suitable input device. External devices 28 can also include portable computer readable storage media such as, for example, thumb drives, portable optical or magnetic disks, and memory cards.

Software and data used to practice embodiments of the present invention can be stored on such portable computer readable storage media and can be loaded onto persistent storage 18 via I/O interface(s) 22. I/O interface(s) 22 may also connect to a display 30. Display 30 provides a mechanism to display data to a user and may be, for example, a computer monitor.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

The health information may be stored within any conventional or other data structures (e.g., files, arrays, lists, stacks, queues, records, etc.) and may be stored in any desired storage unit (e.g., database, data or other repositories, queue, etc.) The health information transmitted from client device 105 to server 150 and from server 150 to requester device 140 may include any desired format and arrangement, and may include any quantity of any types of fields of any size to store the data. The definition and data model for the health information or messages containing the health information may indicate the overall structure in any desired fashion (e.g., computer-related languages, graphical representation, listing, etc.).

The health information may include any information collected by client device 105, any information derived by analyzing information collected by client device 105, and any information otherwise provided to client device 105, requester device 140, and server 150. The health information may include any desired format and arrangement, and may include any quantity of any types of fields of any size to store any desired data. The fields may indicate the presence, absence, actual values, or any other desired characteristics of the data of interest (e.g., quantity, value ranges, etc.). Health information may include all or any desired portion (e.g., any quantity of specific fields) of personal information (PI) or other data of interest within a given implementation or system. The health information may indicate the overall record structure in any desired fashion (e.g., computer-related languages, graphical representation, listing, etc.). The fields for the health information may be selected automatically (e.g., based on metadata, common or pre-defined models or structures, etc.) or manually (e.g., pre-defined, etc.) in any desired fashion for a particular implementation or system. Metadata (e.g., for field selection, common model, etc.) may include any suitable information providing a description of fields or information (e.g., description of content, data type, etc.).

The health information may include any measurable information collected from an organism by any collection device (e.g., sensor, transducer, etc.), any combination of measurable information, and any information derived from analyzing collected information.

The present invention embodiments may employ any number of any type of user interface (e.g., Graphical User Interface (GUI), command-line, prompt, etc.) for obtaining or providing information (e.g., health information and metadata), where the interface may include any information arranged in any fashion. The interface may include any number of any types of input or actuation mechanisms (e.g., buttons, icons, fields, boxes, links, etc.) disposed at any locations to enter/display information and initiate desired actions via any suitable input devices (e.g., mouse, keyboard, etc.). The interface screens may include any suitable actuators (e.g., links, tabs, etc.) to navigate between the screens in any fashion.

The present invention embodiments are not limited to the specific tasks or algorithms described above, but may be utilized for generation and analysis of various types of data, even in the absence of that data. For example, present invention embodiments may be utilized for any types of data interest (e.g, sensitive data (personal information (PI) including information pertaining to patients, customers, suppliers, citizens, and/or employees, etc.) non-sensitive data, data that may become unavailable (e.g., data that is subject to deletion after retention for a minimum time interval (e.g., information subject to various regulations, etc.), information that becomes unavailable due to system outage, power failure, or other data loss, etc.), etc.). Further, present invention embodiments may generate and utilize any quantity of health information for a data construct containing data of interest. The health information may be utilized in the presence or absence of the associated data (e.g., prior to or subsequent to deletion of the data, etc.).

It will be appreciated that the embodiments described above and illustrated in the drawings represent only a few of the many ways of implementing embodiments for context-based access to health information.

The environment of the present invention embodiments may include any number of computer or other processing systems (e.g., client or end-user systems, server systems, etc.) and databases or other repositories arranged in any desired fashion, where the present invention embodiments may be applied to any desired type of computing environment (e.g., cloud computing, client-server, network computing, mainframe, stand-alone systems, etc.). The computer or other processing systems employed by the present invention embodiments may be implemented by any number of any personal or other type of computer or processing system (e.g., desktop, laptop, PDA, mobile devices, etc.), and may include any commercially available operating system and any combination of commercially available and custom software (e.g., browser software, communications software, server software, contextual health information sharing software, health tracking software, etc.). These systems may include any types of monitors and input devices (e.g., keyboard, mouse, voice recognition, etc.) to enter and/or view information.

It is to be understood that the software (e.g., contextual health information sharing software, health tracking software, etc.) of the present invention embodiments may be implemented in any desired computer language and could be developed by one of ordinary skill in the computer arts based on the functional descriptions contained in the specification and flow charts illustrated in the drawings. Further, any references herein of software performing various functions generally refer to computer systems or processors performing those functions under software control. The computer systems of the present invention embodiments may alternatively be implemented by any type of hardware and/or other processing circuitry.

The various functions of the computer or other processing systems may be distributed in any manner among any number of software and/or hardware modules or units, processing or computer systems and/or circuitry, where the computer or processing systems may be disposed locally or remotely of each other and communicate via any suitable communications medium (e.g., LAN, WAN, Intranet, Internet, hardwire, modem connection, wireless, etc.). For example, the functions of the present invention embodiments may be distributed in any manner among the various end-user/client and server systems, and/or any other intermediary processing devices. The software and/or algorithms described above and illustrated in the flow charts may be modified in any manner that accomplishes the functions described herein. In addition, the functions in the flow charts or description may be performed in any order that accomplishes a desired operation.

The software of the present invention embodiments (e.g., contextual health information sharing software, health tracking software, etc.) may be available on a non-transitory computer useable medium (e.g., magnetic or optical mediums, magneto-optic mediums, floppy diskettes, CD-ROM, DVD, memory devices, etc.) of a stationary or portable program product apparatus or device for use with stand-alone systems or systems connected by a network or other communications medium.

The communication network may be implemented by any number of any type of communications network (e.g., LAN, WAN, Internet, Intranet, VPN, etc.). The computer or other processing systems of the present invention embodiments may include any conventional or other communications devices to communicate over the network via any conventional or other protocols. The computer or other processing systems may utilize any type of connection (e.g., wired, wireless, etc.) for access to the network. Local communication media may be implemented by any suitable communication media (e.g., local area network (LAN), hardwire, wireless link, Intranet, etc.).

The system may employ any number of any conventional or other databases, data stores or storage structures (e.g., files, databases, data structures, data or other repositories, etc.) to store information (e.g., health information). The database system may be implemented by any number of any conventional or other databases, data stores or storage structures (e.g., files, databases, data structures, data or other repositories, etc.) to store information (e.g., health information). The database system may be included within or coupled to the server and/or client systems. The database systems and/or storage structures may be remote from or local to the computer or other processing systems, and may store any desired data (e.g., health information).

The present invention embodiments may employ any number of any type of user interface (e.g., Graphical User Interface (GUI), command-line, prompt, etc.) for obtaining or providing information (e.g., health information), where the interface may include any information arranged in any fashion. The interface may include any number of any types of input or actuation mechanisms (e.g., buttons, icons, fields, boxes, links, etc.) disposed at any locations to enter/display information and initiate desired actions via any suitable input devices (e.g., mouse, keyboard, etc.). The interface screens may include any suitable actuators (e.g., links, tabs, etc.) to navigate between the screens in any fashion.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises”, “comprising”, “includes”, “including”, “has”, “have”, “having”, “with” and the like, when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

1. A computer-implemented method of processing requests for health information comprising:
receiving a request for health information of an entity from a requester, wherein a set of access restrictions is associated with the health information;
determining a context of the request based on one or more factors; and
processing the request to retrieve health information appropriate for the requester based on the associated set of access restrictions and the determined context of the request.

2. The computer-implemented method of claim 1, wherein the health information includes one or more from a group of: heart rate, blood pressure, stress level, a quantity of steps, an emotional state, oxygen saturation, temperature, and sleep information.

3. The computer-implemented method of claim 2, wherein the health information is collected by a wearable sensing device.

4. The computer-implemented method of claim 1, wherein the set of access restrictions restricts access to one or more elements of the health information based on one or more from a group of: an identity of the requester, a time interval in which the request is received, a location of one or more of the requester and the entity, and permissions for requesters to access specific elements of the health information.

5. The computer-implemented method of claim 1, wherein the one or more factors for determining the context of the request include one or more from a group of: a location of the entity, an activity performed by the entity, a time of the request, and a relationship of the requester to the entity.

6. The computer-implemented method of claim 1, further comprising:
sending a response to the requester, wherein the response comprises the retrieved health information.

7. A computer system for processing requests for health information, the computer system comprising:
one or more computer processors;
one or more computer readable storage media;
program instructions stored on the one or more computer readable storage media for execution by at least one of the one or more computer processors, the program instructions comprising instructions for:
receiving a request for health information of an entity from a requester, wherein a set of access restrictions is associated with the health information;
determining a context of the request based on one or more factors; and
processing the request to retrieve health information appropriate for the requester based on the associated set of access restrictions and the determined context of the request.

8. The computer system of claim 7, wherein the health information includes one or more from a group of: heart rate, blood pressure, stress level, a quantity of steps, an emotional state, oxygen saturation, temperature, and sleep information.

9. The computer system of claim 8, wherein the health information is collected by a wearable sensing device.

10. The computer system of claim 7, wherein the set of access restrictions restricts access to one or more elements of the health information based on one or more from a group of: an identity of the requester, a time interval in which the request is received, a location of one or more of the requester and the entity, and permissions for requesters to access specific elements of the health information.

11. The computer system of claim 7, wherein the one or more factors for determining the context of the request include one or more from a group of: a location of the entity, an activity performed by the entity, a time of the request, and a relationship of the requester to the entity.

12. The computer system of claim 7, further comprising instructions for:
sending a response to the requester, wherein the response comprises the retrieved health information.

13. A computer program product for processing requests for health information, the computer program product comprising a computer readable storage medium having program instructions embodied therewith, the program instructions executable by a computer to cause the computer to:
receiving a request for health information of an entity from a requester, wherein a set of access restrictions is associated with the health information;
determining a context of the request based on one or more factors; and
processing the request to retrieve health information appropriate for the requester based on the associated set of access restrictions and the determined context of the request.

14. The computer program product of claim 13, wherein the health information includes one or more from a group of: heart rate, blood pressure, stress level, a quantity of steps, an emotional state, oxygen saturation, temperature, and sleep information.

15. The computer program product of claim 14, wherein the health information is collected by a wearable sensing device.

16. The computer program product of claim 13, wherein the set of access restrictions restricts access to one or more elements of the health information based on one or more from a group of: an identity of the requester, a time interval in which the request is received, a location of one or more of the requester and the entity, and permissions for requesters to access specific elements of the health information.

17. The computer program product of claim 13, wherein the one or more factors for determining the context of the request include one or more from a group of: a location of the entity, an activity performed by the entity, a time of the request, and a relationship of the requester to the entity.

18. The computer program product of claim 13, further comprising instructions for:
sending a response to the requester, wherein the response comprises the retrieved health information.
